(19) United States
(12) Patent Application Publication
(10) Pub. No.: US 2007/0233184 A1
(43) Pub. Date: Oct. 4, 2007

(54) Medical instrument for grasping surgical implant rods

(75) Inventor: Kingston Wong, Beach Park, IL (US)

Correspondence Address: COOK, ALEX, MCFARRON, MANZO, CUMMINGS & MEHLER LTD, SUITE 2850, 200 WEST ADAMS STREET, CHICAGO, IL, 60606, US

(73) Assignee: Klein Tools, Inc.

(21) Appl. No.: 11/363,900

(22) Filed: Feb. 28, 2006

(51) Int. Cl. A61B 17/00

(52) U.S. Cl. 606/205

(57) ABSTRACT

A medical instrument is provided for grasping a surgical implant rod. The medical instrument includes a first jaw including a proximal and a distal end. A handle portion is carried by the proximal end of the first jaw. A rod abutment portion is carried by the distal end of the first jaw. A slidably movable second jaw extends along the first jaw and carries a rod abutment portion. An operation of the handle portion causes a ratcheting mechanism to move the rod abutment portion of the second jaw toward the abutment portion of the first jaw to grasp the surgical implant rod therebetween.

### TECHNICAL FIELD

The present invention generally relates to a medical instrument for grasping surgical implant rods. More specifically, a medical instrument for grasping a surgical implant rod is provided including a handle portion which causes a ratcheting mechanism to move a rod abutment portion of a second jaw toward a rod abutment portion of a first jaw to grasp the surgical implant rod therebetween.

### BACKGROUND OF THE INVENTION

Various surgeries require the implantation of surgical implant rods to reinforce deficient bone structure in the body. For example, such surgical implant rod implants have been used in neurosurgical procedures to treat curvature, trauma, deformity, and degenerative conditions of the spine. These implants typically include screws capable of supporting alignment of support rods placed bilaterally along the vertebrae. Surgical implant rods may further be used as cross-link members that bridge the sagittal of the spine.

As shown in FIG. 1, an example of a commercial spinal implantation system which includes surgical implant rods is the Legacy™ spinal implantation system manufactured by Medtronic, Inc. The system 10 generally includes a plurality of connection members 12 for supporting alignment rods 14a, b onto the vertebrae 16.

As shown in FIG. 2, Gelbard, U.S. Pat. No. 5,766,254 for a “Spinal Stabilization Implant System,” incorporated herein by reference and made a part hereto, describes a cross-stabilization system 18 for the stabilization of a human spine by fixation of the vertebra 20. The cross-stabilization system 18 specifically includes screws 22 to support alignment rods 24a, b onto the vertebrae 20.

As shown in FIG. 3, Ray et al, U.S. Pat. No. 5,300,073 for a “Sacral Implant System,” incorporated herein by reference and made a part hereto, describes a sacral implant system 28 including first and second sacral plates 30a,b affixed on opposite sides of the sacrum. First and second fixation rods 32a,b extend from the sacral plates 30a,b and are fixed onto various vertebrae via connection members 34 and hooks 36.

As shown in FIGS. 4a, 4b, and 4c, Lin, U.S. Pat. No. 5,176,679 for a “Vertebral Locking and Retrieving System,” incorporated herein by reference and made a part hereto, describes a vertebral locking and retrieving system 38 including at least one lock pin 40 with coupling components 42 for connecting a plurality of locking components 44. The locking components 44 are generally in the form of surgical implant rods which aid in the stabilization of vertebrae 46.

In another example, surgical implant rods have alternatively been implanted into other areas of the body. For example, surgical implant rod implants have been used to treat bone disorders such as fractures. As shown in FIG. 5, Chapman et al., U.S. Pat. No. 5,190,544 for “Modular Femoral Fixation System,” incorporated herein by reference and made a part hereto, describes one such implant system, specifically for use in the treatment of femoral disorders. The '554 implant system 48 includes an intramedullay rod 50 for supporting a fractured femur 52.

Surgical procedures for implanting surgical implant rods require much care. For example, the implantation of surgical implant rods in neurosurgical procedures requires the manipulation of the surgical implant rods so as to not damage the spinal cord, other nerves associated therewith, and surrounding tissue. Accordingly, the user is required to insert the rod into very small incisions, maneuver the rod in very small spaces, and maneuver and rotate the rod around various obstacles.

Current medical instruments for grasping and manipulating surgical implant rods are deficient in their weak and unstable grasp of the rod. Furthermore, current medical instruments require an exorbitant grasping force from the user. This results in sore hands and unreliable manipulation of the instrument by the user during delicate operation thereof.

Accordingly, it is an object of the present invention to provide a medical instrument which firmly grasps and reliably manipulates surgical implant rods with delicate precision and without exorbitant force required by the user.

### SUMMARY OF THE INVENTION

In view of the desired goals of the invention claimed herein, a medical instrument is provided for grasping a surgical implant rod. The medical instrument includes a first jaw including a proximal and a distal end. A handle portion is carried by the proximal end of the first jaw. A rod abutment portion is carried by the distal end of the first jaw. A slidably movable second jaw extends along the first jaw and carries a rod abutment portion. An operation of the handle portion causes a ratcheting mechanism to move the rod abutment portion of the second jaw toward the rod abutment portion of the first jaw to grasp the surgical implant rod therebetween. In another embodiment, the medical instrument further includes an outer reinforcement tube enveloping the first jaw and second jaw.

In yet another embodiment, the handle portion includes a pair of legs. One of the one of the legs may be pivotably movable such that a pivot of the leg causes the ratcheting mechanism to move the rod abutment portion of the second jaw toward the rod abutment portion of the first jaw to grasp the surgical implant rod therebetween. Alternatively, both of the legs are pivotably movable such that a pivot of both legs causes the ratcheting mechanism to move the rod abutment portion of the second jaw toward the rod abutment portion of the first jaw to grasp the surgical implant rod therebetween. Moreover, a pair of leaf springs may be connected between the pair of legs for biasing the legs away from one another.

In yet another embodiment, a lock lever is provided associated with the ratcheting mechanism. The lock lever biases the ratcheting mechanism in a locked position. Engagement of the lock lever unlocks the ratcheting mechanism and allows the movement of the rod abutment portion of the second jaw away from the rod abutment portion of the first jaw.

It should be understood that the present invention includes a number of different aspects or features which may have utility alone and/or in combination with other aspects or features. Accordingly, this summary is not exhaustive identification of each such aspect or feature that is now or may hereafter be claimed, but represents an overview of certain aspects of the present invention to assist in understanding the more detailed description that follows. The scope of the invention is not limited to the specific embodiments described below, but is set forth in the claims now or hereafter filed.

### BRIEF DESCRIPTION OF THE SEVERAL VIEWS OF THE DRAWINGS

Throughout this description, reference has been and will be made to the accompanying views of the drawing wherein like subject matter has like reference numerals, and wherein:

FIG. 1 is a perspective view of the prior art Legacy™ spinal implantation system manufactured by Medtronic, Inc. including surgical implant rods;

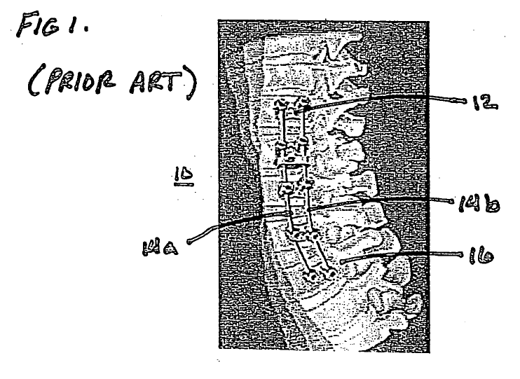

FIG. 2 is a perspective view of a prior art cross-stabilization system including surgical implant rods;

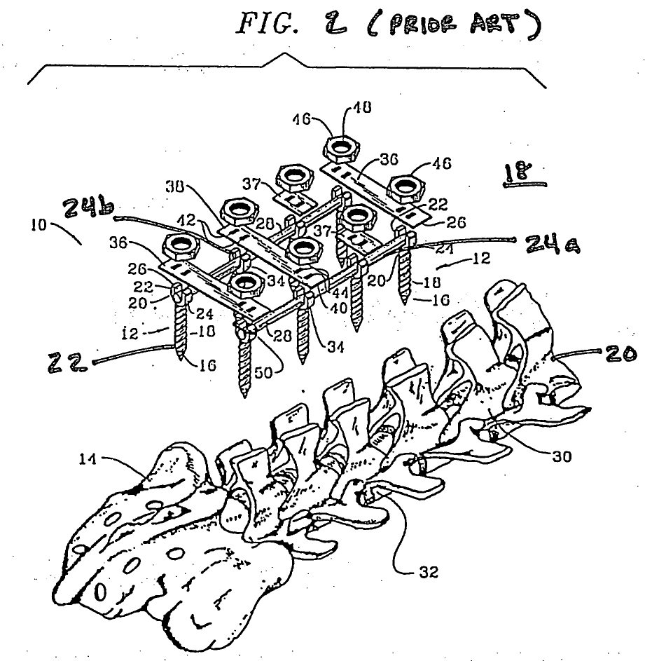

FIG. 3 is a perspective view of a prior art sacral implant system including surgical implant rods;

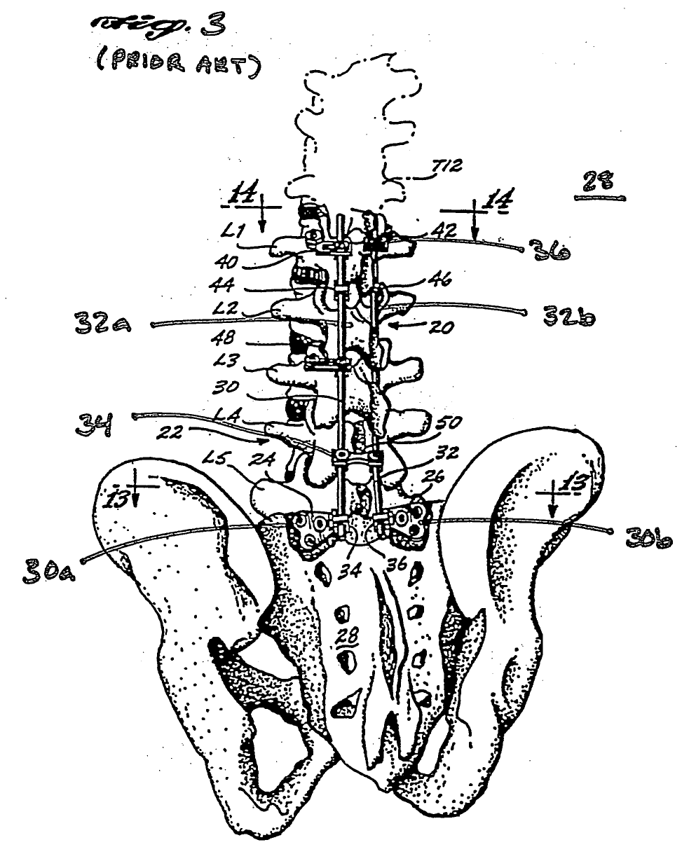

FIGS. 4a, 4b, and 4c are perspective views of a prior art vertebral locking and retrieving system including surgical implant rods;

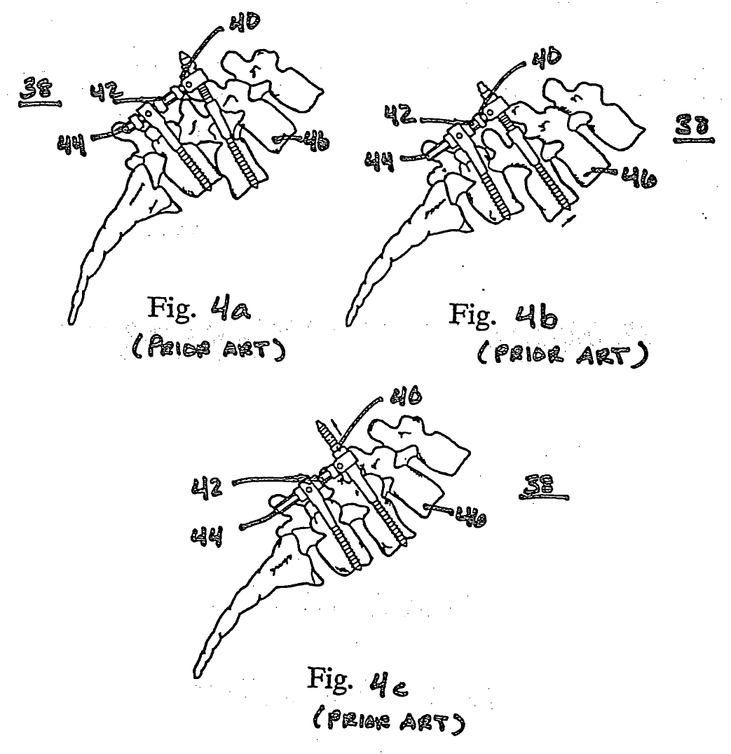

FIG. 5 is a perspective view of a prior art femoral implant system including surgical implant rods;

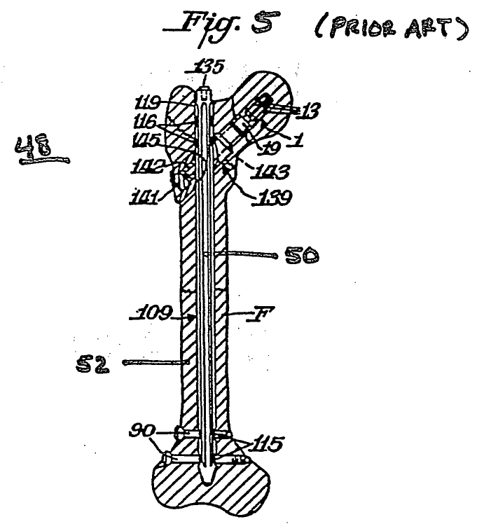

FIG. 6 is an exploded view of a first embodiment medical instrument for grasping surgical implant rods in accordance with the teachings of the present invention;

FIG. 7 is a perspective view of the first embodiment medical instrument of FIG. 6 in an ungrasping position;

FIG. 8 is a perspective view of the first embodiment medical instrument of FIG. 6 in a grasping position;

FIG. 9 is a cross-sectional view of the first embodiment medical instrument of FIG. 6 illustrating the ratcheting mechanism and associated parts;

FIG. 10 is another cross-sectional view of the first embodiment medical instrument of FIG. 6 illustrating the ratcheting mechanism and associated parts;

FIG. 11 is a second embodiment medical instrument for grasping surgical implant rods including attachable rod abutment portions in accordance with the teachings of the present invention;

FIG. 12 is a third embodiment medical instrument for grasping surgical implant rods including a fixed handle and a contoured handle in accordance with the teachings of the present invention; and

FIGS. 13a and 13b are isometric views of another embodiment flat spring for use with a medical instrument for grasping surgical implant rods in accordance with the teachings of the present invention.

### DETAILED DESCRIPTION OF THE MULTIPLE EMBODIMENTS

In accordance with the teachings of the present invention, a medical instrument is provided for grasping a surgical implant rod. The medical instrument is preferably constructed of a durable material that can be autoclaved at a high temperature and pressure. The medical instrument preferably further has a non-glare surface so as to not interfere with the user's sight while using the instrument. For example, the medical instrument may be constructed of a stainless metal material. More specifically, the medical instrument may be constructed of a titanium alloy.

While the invention is susceptible of embodiment in many different forms and in various combinations, particular focus will be on the multiple embodiments of the invention described herein with the understanding that such embodiments are to be considered exemplifications of the principles of the invention and are not intended to limit the broad aspect of the invention.

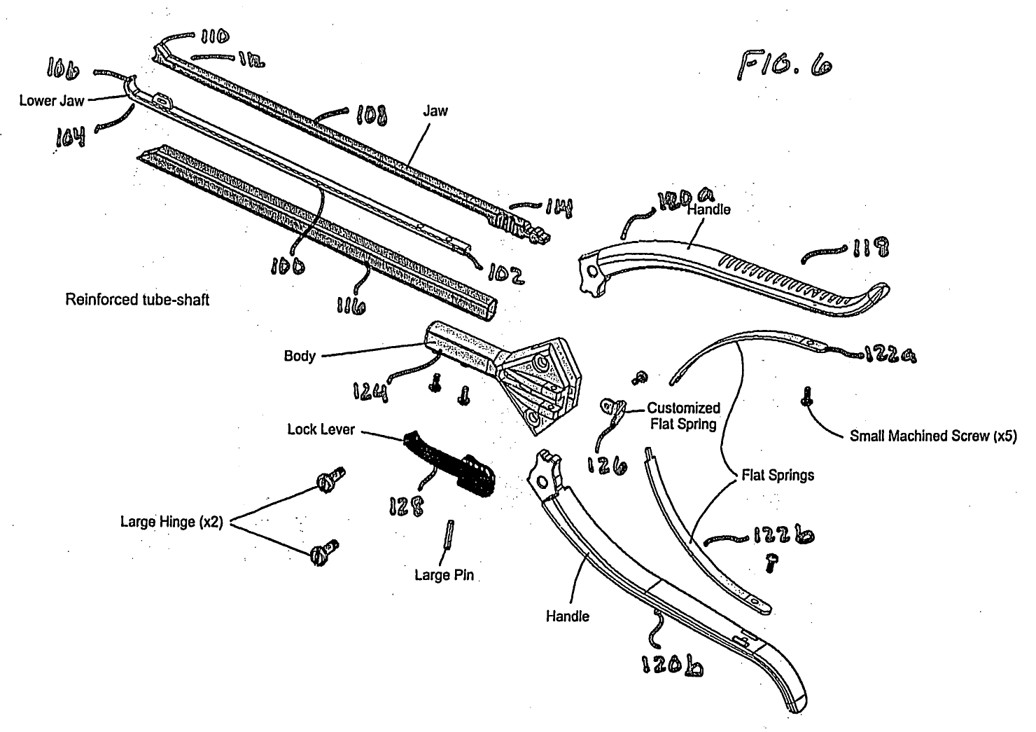

As shown in FIG. 6, in accordance with a first aspect of the present invention, the medical instrument includes a first jaw 100 including a proximal end 102 and a distal end 104. A rod abutment portion 106 is carried by the distal end 104 of the first jaw 100. A second jaw 108 is further provided which carries a rod abutment portion 110 on its distal end 112 and a ratcheting mechanism engagement portion 114 on its proximal end. An outer reinforcement tube 116 for enveloping the first jaw 100 and second jaw 108 is further provided.

A handle portion 118 is further provided comprising a pair of legs 120a,b with associated leaf springs 122a,b. A ratcheting mechanism 124 is further provided with a flat spring 126 and a lock lever 128 associated therewith.

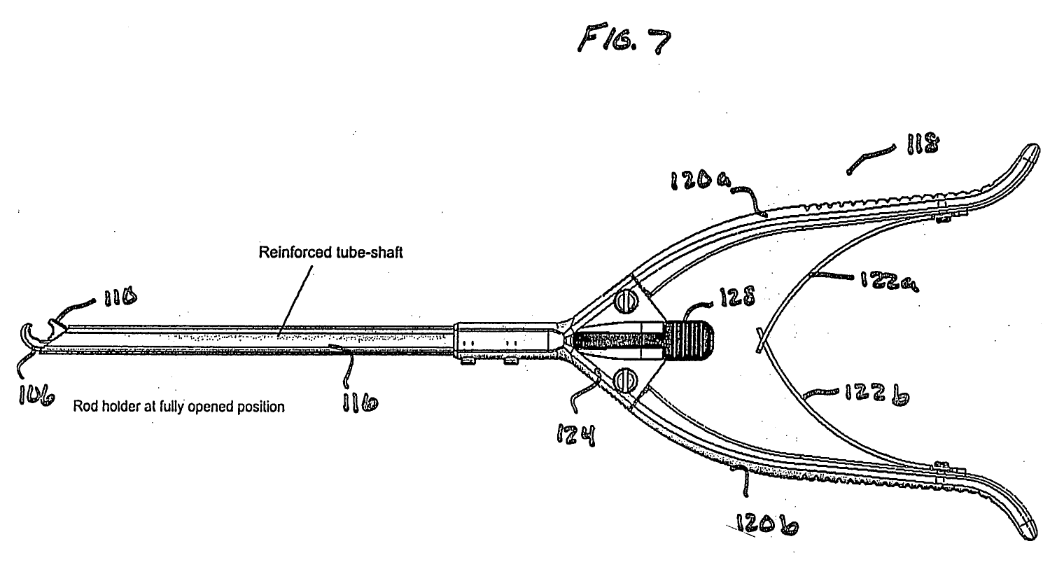

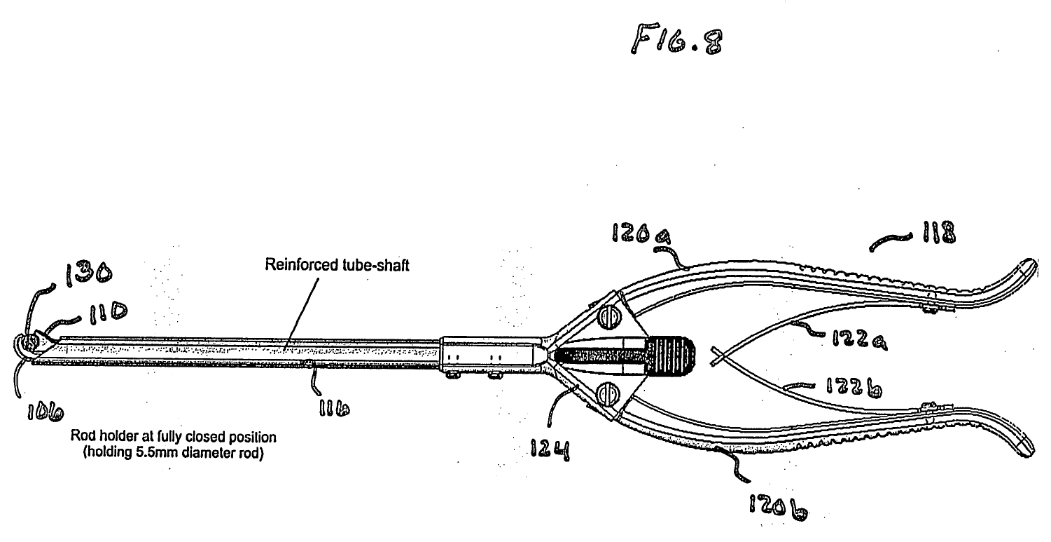

FIGS. 7 and 8 generally illustrate the operation of the first embodiment medical instrument of FIG. 6. FIG. 7 is a perspective view of the first embodiment medical instrument of in an ungrasping position, whereas FIG. 8 is a perspective view of the first embodiment medical instrument in a grasping position.

The medical instrument includes an outer reinforcement tube 116 enveloping the first jaw and second jaw. This reinforcement tube 116 strengthens the elongated first jaw and second jaw structure, thereby allowing the user to increase stress, strain, and torque on the elongated structure and the medical instrument. It further protects the first and second jaws from exposure to the environment. In yet another benefit, the reinforcement tube 116 protects the patient from any unintentional pinching of nerves, tissue, etc. between the first and second jaws.

The handle portion 118 is carried by the proximal end of the first jaw. The pair of leaf springs 122a,b is connected between the pair of legs 120a,b for biasing the legs away from one another. An operation of the handle portion 118 causes the ratcheting mechanism 124 to move the rod abutment portion 110 of the second jaw toward the rod abutment portion 106 of the first jaw to grasp the surgical implant rod 130 therebetween. More specifically, both of the legs 120a,b are pivotably movable such that a pivot of both legs toward each other (e.g, a gripping action by the user) causes the ratcheting mechanism 124 to operate.

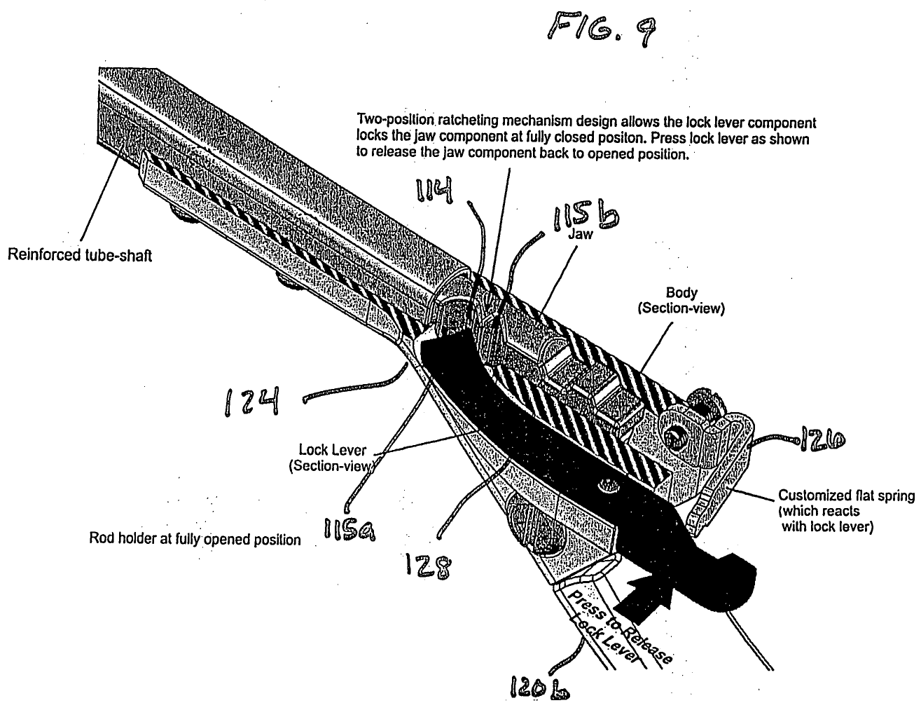

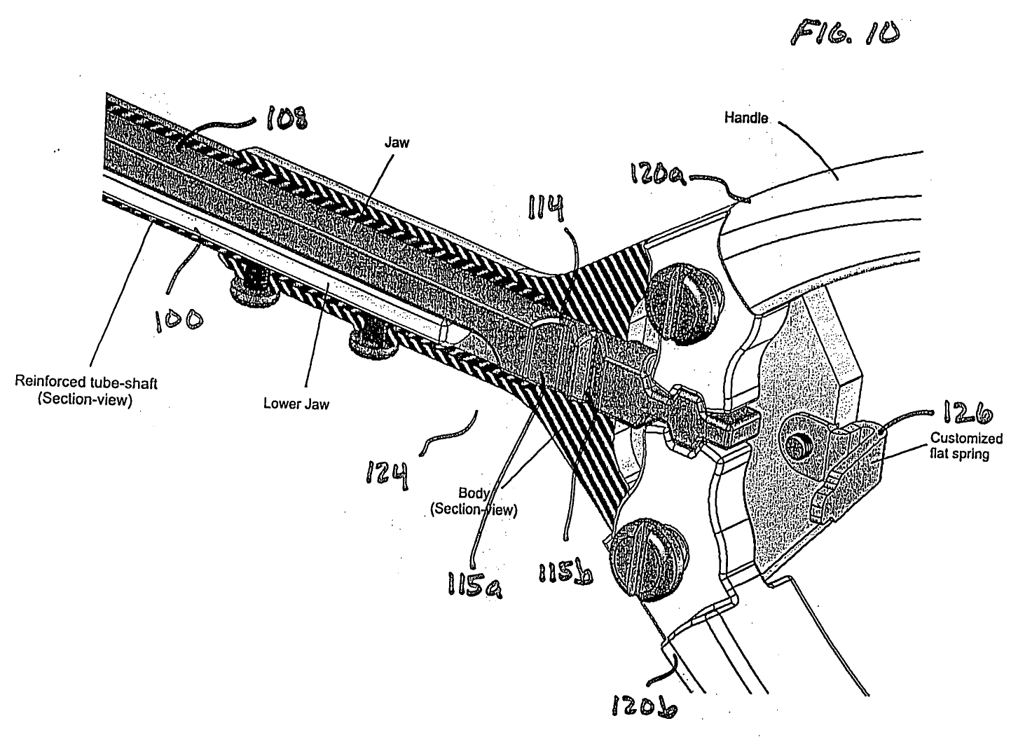

Turning specifically to FIGS. 9 and 10 for illustration of the operation of the ratcheting mechanism 124 and associated structures, the legs 120a,b are connected to the proximal end of the second jaw 108. As the legs 120a,b are pivoted toward each other (e.g, a gripping action by the user) the ratcheting mechanism engagement portion 114 is slidably moved distally such that the lock lever 128 advances from an initial position situated at the first notch 115a of the ratcheting mechanism engagement portion 114 to a second position situated at the second notch 115b of the ratcheting mechanism engagement portion. In this way, the rod abutment portion of the second jaw 108 moves toward the rod abutment portion of the first jaw 100.

The lock lever 128 is biased to lock the second jaw 108 in place via flat spring 126. In order to release the grasp on the rod 130, the distal part of the lock lever is pressed (e.g., through pressing by the user's thumb) so that the ratcheting mechanism engagement portion is allowed to move back to the initial position (first notch 115a) from the second position (second notch 115b). Without deviating from the spirit of the present invention, it is contemplated that multiple notches may be included on the ratcheting mechanism engagement portion so that there are many different positions and, therefore, many different sizes of rods that may be grasped.

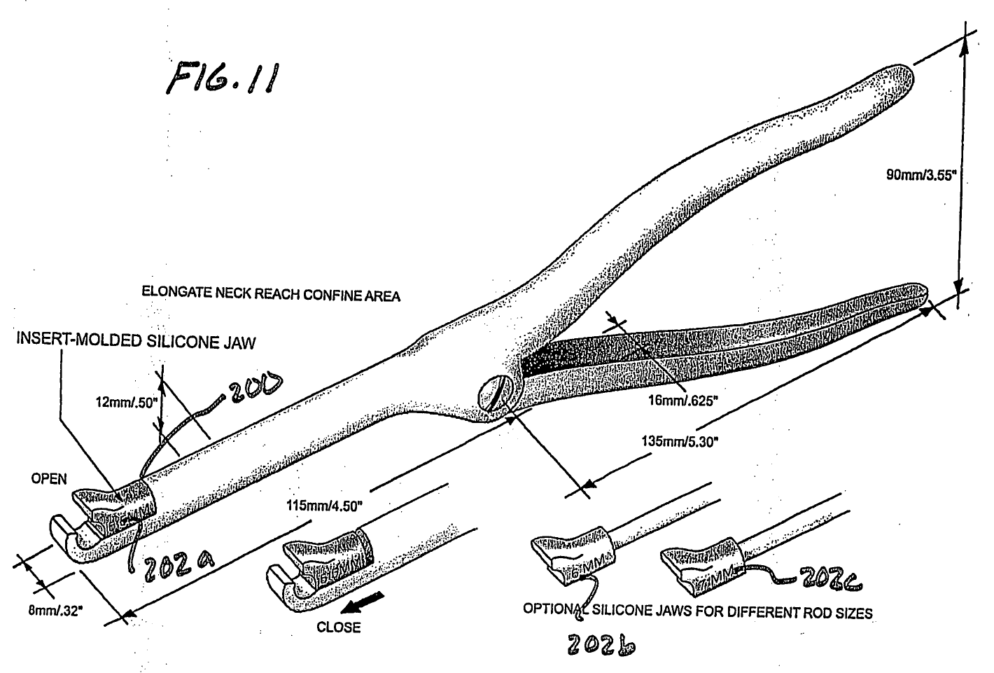

In yet another embodiment and now turning to FIG. 11, the second jaw 200 may include attachable or detachable rod abutment portions 202a, b, c. In this embodiment, the rod abutment portions 202a, b, c are sized such that many different sizes of rods can be accommodated. Without deviating from the spirit of the present invention, it is contemplated that many different shapes of rod abutment portions of either the first or second jaw may be implemented. For example, the rod abutment portion may be shaped to complement generally triangular, elliptical, rectangular, trapezoidal, square, etc. rods.

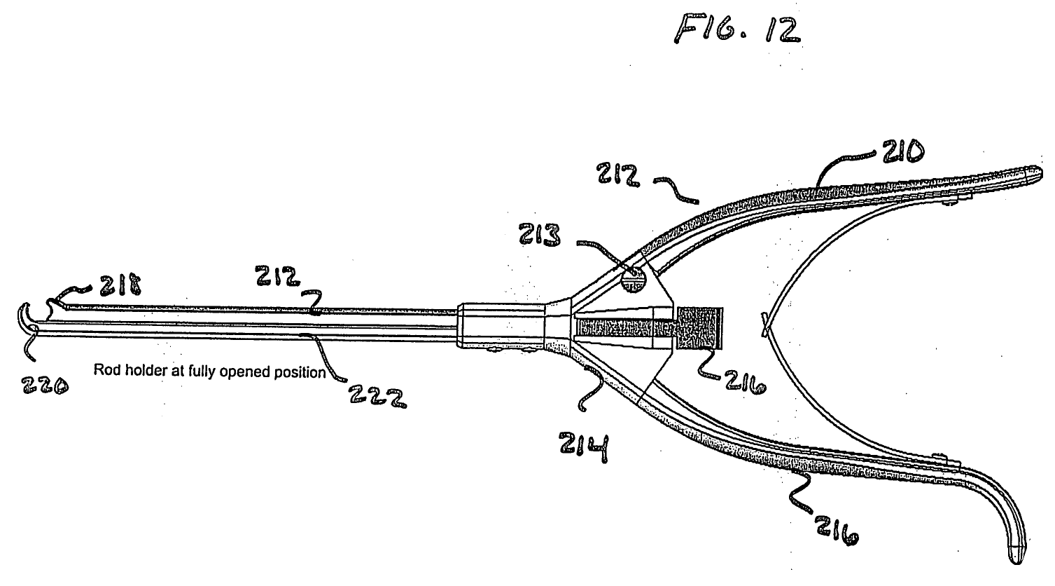

In yet another embodiment and now turning to FIG. 12, only the upper leg 210 of the handle 212 may be pivoted, as evident by a screw 213 in the ratcheting mechanism 214. In this embodiment, only the upper leg 210 is connected to the second jaw 212 via the ratcheting mechanism engagement portion. As the upper leg 210 is pivoted toward the fixed lower leg 216 (e.g, a gripping action by the user) the ratcheting mechanism engagement portion is slidably moved distally such that the lock lever 216 advances from an initial position to another position. In this way, the rod abutment portion of the second jaw 218 moves toward the rod abutment portion 220 of first jaw 222. Moreover, as illustrated in FIG. 12, the contour of the legs of the handle may be adapted for a better ergonomic fit with the hand. In this embodiment, the lower leg 210 of the handle 212 is substantially curved. It is further illustrated in this embodiment without an outer reinforcement tube for enveloping the first jaw 222 and second jaw 218.

Furthermore, without deviating from the spirit of the present invention, it is contemplated that many different types of handles may be used as long as they work with a ratcheting mechanism. For example, the handle may include a rotatable shaft which is associated with a jaw and a ratcheting mechanism, wherein the rotation of the handle causes the ratcheting mechanism to move the rod abutment portion of jaw toward the rod abutment portion of another jaw to grasp a surgical implant rod therebetween.

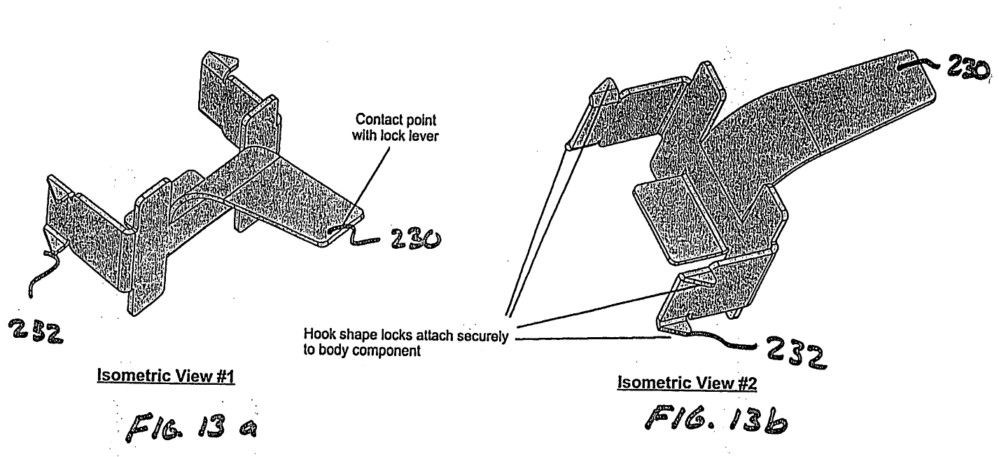

In yet another embodiment and now turning to FIG. 13, a flat spring is illustrated having a more substantial contact spring. The spring includes a more substantial contact point 230 with the lock lever. Hook shape locks 232 attach the flat spring more securely to the ratcheting mechanism.

While this invention has been described with reference to certain illustrative aspects, it will be understood that this description shall not be construed in a limiting sense. Rather, various changes and modifications can be made to the illustrative embodiments without departing from the true spirit, central characteristics and scope of the invention, including those combinations of features that are individually disclosed or claimed herein. Furthermore, it will be appreciated that any such changes and modifications will be recognized by those skilled in the art as an equivalent to one or more elements of the following claims, and shall be covered by such claims to the fullest extent permitted by law.

1. A medical instrument for grasping a surgical implant rod, comprising:
a first jaw including a proximal and a distal end;
a first rod abutment portion carried by the distal end of said first jaw;
a handle portion carried by the proximal end of the first jaw
a slidably movable second jaw extending along the first jaw,
a second rod abutment portion carried by said slidably movable second jaw; and
a ratcheting mechanism associated with the second jaw and the handle potion; wherein an operation of said handle portion causes said ratcheting mechanism to move the rod abutment portion of said second jaw toward the rod abutment portion of said first jaw to grasp the surgical implant rod therebetween.

2. The medical instrument for grasping a surgical implant rod of claim 1, an outer reinforcement tube enveloping said first jaw and second jaw.

3. The medical instrument for grasping a surgical implant rod of claim 1, wherein the handle portion includes a pair of legs.

4. The medical instrument for grasping a surgical implant rod of claim 3, wherein one of the legs is pivotably movable such that a pivot of the leg causes said ratcheting mechanism to move the rod abutment portion of said second jaw toward the rod abutment portion of said first jaw to grasp the surgical implant rod therebetween.

5. The medical instrument for grasping a surgical implant rod of claim 3, wherein one of the legs is substantially fixed.

6. The medical instrument for grasping a surgical implant rod of claim 3, wherein both of the legs are pivotably movable such that a pivot of both legs causes said ratcheting mechanism to move the rod abutment portion of said second jaw toward the rod abutment portion of said first jaw to grasp the surgical implant rod therebetween.

7. The medical instrument for grasping a surgical implant rod of claim 3, further comprising a pair leaf springs connected between the pair of legs for biasing the legs away from one another.

8. The medical instrument for grasping a surgical implant rod of claim 1, further comprising a lock lever associated with said ratcheting mechanism for locking the ratcheting mechanism in a selected position.

9. The medical instrument for grasping a surgical implant rod of claim 8, further comprising a flat spring for biasing the lock lever in a locked position.

10. The medical instrument for grasping a surgical implant rod of claim 8, wherein engagement of the locked lever allows movement of the rod abutment portion of said second jaw away from the rod abutment portion of said first jaw.

11. The medical instrument for grasping a surgical implant rod of claim 1, wherein the medical instrument is constructed of a stainless metal material.

12. The medical instrument for grasping a surgical implant rod of claim 1, wherein the medical instrument is constructed of titanium alloy.

13. The medical instrument for grasping a surgical implant rod of claim 1, further comprising a ratcheting mechanism engagement portion carried by the proximal end of the slidably moveable second jaw, wherein an operation of said handle portion causes said ratcheting mechanism to engage said ratcheting mechanism engagement portion to move the rod abutment portion of said second jaw toward the rod abutment portion of said first jaw.

14. The medical instrument for grasping a surgical implant rod of claim 13, wherein the ratcheting mechanism engagement portion includes a first notch position and a second notch position, wherein an operation of said handle portion causes said ratcheting mechanism to engage said ratcheting mechanism engagement portion to move the rod abutment portion of said second jaw toward the rod abutment portion of said first jaw from the first notch position to the second notch position.

15. The medical instrument for grasping a surgical implant rod of claim 1, wherein the second rod abutment portion is detachable.

16. The medical instrument for grasping a surgical implant rod of claim 1, wherein the handle is ergonomically contoured.
